(19) United States
(12) Patent Application Publication
(10) Pub. No.: US 2024/0417290 A1
(43) Pub. Date: Dec. 19, 2024

(54) Compositions and Methods for Flocculation

(71) Applicant: Locus Solutions IPCo, LLC, Solon, OH (US)

(72) Inventor: Gabriela KNESEL, Solon, OH (US)

(21) Appl. No.: 18/698,475

(22) PCT Filed: Jun. 7, 2023
(86) PCT No.: PCT/US2023/068031

§ 371 Date: Apr. 4, 2024

Related U.S. Application Data
(60) Provisional application No. 63/350,213, filed on Jun. 8, 2022.

(51) Int. Cl. C02F 1/52; C02F 1/00; C02F 103/10

(52) U.S. Cl. CPC C02F 1/5272; C02F 1/5263; C02F 2001/007; C02F 2103/10

(57) ABSTRACT

The subject invention provides safe, environmentally-friendly compositions and efficient methods for flocculation. More specifically, the subject invention provides compositions derived from microorganisms for flocculation, which can be used for increasing the rate of flocculation and/or the amount of agglomerated particles.

### CROSS-REFERENCE TO RELATED APPLICATION

This application claims priority to U.S. Provisional Patent Application No. 63/350,213, filed Jun. 8, 2022, which is incorporated herein by reference in its entirety.

### BACKGROUND OF THE INVENTION

Efficiently removing deposits from the earth and reducing pollution associated with various mining and quarrying practices commonly employs the use of flocculants. During mining, flocculants can be added to adsorb a target mineral through electrostatic interactions, hydrogen bonding, or chemical bonding. Currently, starch and benzoyl hydroxamic acid are used as a flocculant in iron ore mining to flocculate hematite and other low-grade rare earth elements, minerals, or metals.

Mining or excavating of rock, including quarrying, can also result in water pollution. During mining or quarrying, rock can be exposed to the atmosphere, including wind and rain, which can result in oxidation of the minerals to toxic chemicals, such as, for example, cyanide, sulfuric acid and soluble iron. Water draining from coal mining tailings and other sources, including iron sulfide and related sulfide minerals, is commonly referred to as “acid mine drainage” or “acid rock drainage.” In addition to sulfuric acid and soluble iron, acid mine drainage can have high concentrations of heavy metals such as, for example, molybdenum, tungsten, chromium, manganese, nickel, arsenic, and vanadium. As well as wastewater derived from coal mines, silver, gold and copper mines can also produce water drainage containing toxic dissolved metals. The toxic water can contaminate groundwater and aquifers.

A number of processes have been employed for treating mine drainage by neutralizing or otherwise addressing the drainage. For example, acid mine drainage can be treated with bases such as calcium carbonate, calcium oxide, calcium hydroxide, sodium hydroxide, sodium carbonate, and ammonia. In addition to chemical treatments, acid mine drainage has been treated using a variety of biological treatment schemes. Biological mechanisms typically rely on the construction of containment ponds and the like to subject the acid mine drainage to extended biological action.

Currently, commonly employed methods of acid mine treatment involve chemical precipitation of metals by increasing the pH and/or adding flocculants to the discharge water or wastewater to form metal hydroxides. This is accomplished by adding flocculants, such as, for example various forms of lime (e.g., calcium carbonate and calcium oxide), whereby the increasing pH of water results in metal hydroxide precipitation. However, some mining sites have low concentrations of iron or other compounds, so in order to flocculate and/or precipitate the metal hydroxides, large amounts of lime and other compounds used for the treatment of water drainage or wastewater from mines must be added. The use of the excess amount of the flocculants reduces the efficiency of water treatment and increase the costs.

In addition to the use of flocculants for mining, a flocculent is used in paper making, beer brewing, and for treating sewage sludge from municipal wastewater or stormwater. In recent years, the amount of generated sludge is increasing, and the properties of the sludge are becoming worse. However, with conventional flocculants, the amount of treatable sludge is limited, and the treatment conditions are not satisfactory in terms of the water content of a dewatered cake, the recovery rate, and the removability of a cake from filter cloth.

Therefore, novel, improved flocculating compositions and methods are needed.

### BRIEF SUMMARY OF THE INVENTION

The subject invention relates generally to flocculant compositions and methods of using these compositions. More specifically, the subject invention provides environmentally-friendly flocculant compositions and methods for flocculation, such as, for example, methods of mining, wastewater treatment, paper production, and beer brewing. In certain embodiments, existing methods can incorporate the subject compositions and methods.

Advantageously, the compositions and methods of the subject invention increase the efficiency of flocculation and can decrease the chemical usage, including chemical surfactant usage, required for flocculation. Accordingly, the subject invention can be useful for reducing the time needed for mining, water treatment (e.g., mining wastewater, municipal wastewater, stormwater, swimming pool water) or production of various products, including, for example, paper and beer.

In certain embodiments, the subject invention provides compositions comprising components that are derived from microorganisms. In certain embodiments, the composition comprises a microbial biosurfactant. In certain embodiments, the composition comprises one or more biosurfactants, and, optionally, other compounds, such as, for example, water; chemical surfactants; polymers; pH adjusters; water softeners; ion exchangers; corrosion inhibitors; algaecides; biocides; clarifying agents; coagulants; inorganic salts, including, aluminum, iron, magnesium, and calcium salts; or any combination thereof.

In certain embodiments, the biosurfactant of the composition is utilized in crude form. The crude form can comprise, in addition to the biosurfactant, fermentation broth in which a biosurfactant-producing microorganism was cultivated, residual microbial cell matter or live or inactive microbial cells, residual nutrients, and/or other microbial growth by-products.

In some embodiments, the biosurfactant is utilized after being extracted from a fermentation broth and, optionally, purified.

The biosurfactant according to the subject invention can be a glycolipid (e.g., sophorolipids, rhamnolipids, cellobiose lipids, mannosylerythritol lipids and trehalose lipids), lipopeptide (e.g., surfactin, iturin, fengycin, arthrofactin, and lichenysin), flavolipid, phospholipid (e.g., cardiolipins), fatty acid ester compound, fatty acid ether compound, and/or high molecular weight polymers such as lipoproteins, lipopolysaccharide-protein complexes, and polysaccharide-protein-fatty acid complexes.

In certain specific embodiments, the biosurfactant is a sophorolipid (SLP), including linear SLP, lactonic SLP, acetylated SLP, de-acetylated SLP, salt-form SLP, esterified SLP derivatives, amino acid-SLP conjugates, and other SLP derivatives or isomers that exist in nature and/or are produced synthetically. In preferred embodiments, the SLP is a linear SLP or a derivatized linear SLP.

In certain embodiments, the subject invention provides a method for flocculation, wherein the method comprises the following step: contacting a flocculating composition according to the subject invention to a liquid.

In some embodiments, the method enhances or increases the rate of flocculation and/or the amount flocculated particles that can be less than about 1 mm, about 500 μm, about 100 μm, about 10 μm, about 1 μm, about 100 nm, about 10 nm, or about 1 nm in diameter.

In some embodiments, the method comprises contacting a flocculating composition comprising a biosurfactant and, optionally, other components, such as, for example, water, chemical surfactants, polymers, pH adjusters, water softeners, ion exchangers, corrosion inhibitors, algaecides, biocides, clarifying agents, coagulants, or inorganic salts to a liquid. In certain embodiments, the flocculating composition can be applied to the liquid for a period of time and/or until a distinct volume of the composition has been applied. The step can be repeated as many times as necessary to achieve a desired amount of agglomerated particles or rate of agglomeration of particles.

In certain embodiments, the flocculating composition according to the subject invention is effective due to enhancing and/or increasing the rate of agglomeration or total amount of the agglomerated particles from a liquid containing of colloidal suspension of said particles. For example, in some embodiments, a sophorolipid will form a micelle containing, linking, or neutralizing the particles, wherein the micelle is less than 500 μm, less than 100 μm, less than 10 μm, less than 1 μm, less than 100 nm, less than 50 nm, less than 25 nm, less than 15 nm or less than 10 nm in size.

In certain embodiments, the methods of the subject invention result in at least a 25% increase in flocculation of suspended particles, preferably at least a 50% increase, after one treatment. In certain embodiments, the liquid can be treated multiple times to further increase the amount of flocculated particles.

Advantageously, in certain embodiments, the flocculating composition according to the subject invention can be effective at agglomerating toxic suspended particles. Furthermore, the methods of the subject invention do not require complicated equipment or high energy consumption, and production of the composition can be performed on site, for example, at a mine or at a wastewater treatment facility.

### DETAILED DESCRIPTION

The subject invention relates generally to the flocculation of suspended particles in a liquid. More specifically, the subject invention provides environmentally-friendly compositions and methods for flocculation, such as, for example, liquids that are produced at mining sites, wastewater, and water derived from industrial activities. Accordingly, the subject invention is useful for improving the efficiency and efficacy of methods of flocculation. Advantageously, the compositions and methods of the subject invention increase the flocculation of particles using safe, environmentally-friendly compositions.

### Selected Definitions

As used herein, “applying” a composition or product refers to contacting it with a target or site such that the composition or product can have an effect on that target or site. The effect can be due to, for example, microbial growth and/or the action of a biosurfactant or other microbial growth by-product.

As used herein, a “biofilm” is a complex aggregate of microorganisms, such as bacteria, yeast, or fungi, wherein the cells adhere to each other and/or to a surface using an extracellular matrix. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 98%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism or a substance necessary for taking part in a particular metabolic process. A metabolite can be an organic compound that is a starting material, an intermediate in, or an end product of metabolism. Examples of metabolites include, but are not limited to, enzymes, acids, solvents, alcohols, proteins, vitamins, minerals, microelements, amino acids, biopolymers and biosurfactants.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

As used herein, “surfactant” means a compound that lowers the surface tension (or interfacial tension) between two liquids or between a liquid and a solid. Surfactants act as, e.g., detergents, wetting agents, emulsifiers, foaming agents, and/or dispersants. A “biosurfactant” is a surface-active substance produced by a living cell and/or using naturally-derived substrates.

Biosurfactants are a structurally diverse group of surface-active substances consisting of two parts: a polar (hydrophilic) moiety and non-polar (hydrophobic) group. Due to their amphiphilic structure, biosurfactants can, for example, increase the surface area of hydrophobic water-insoluble substances, increase the water bioavailability of such substances, and change the properties of bacterial cell surfaces. Biosurfactants can also reduce the interfacial tension between water and oil and, therefore, lower the hydrostatic pressure required to move entrapped liquid to overcome the capillary effect. Biosurfactants accumulate at interfaces, thus reducing interfacial tension and leading to the formation of aggregated micellar structures in solution. The formation of micelles provides a physical mechanism to mobilize, for example, oil in a moving aqueous phase.

The ability of biosurfactants to reduce the surface tension also permits their use as antibacterial, antifungal, and hemolytic agents to, for example, control pests and/or microbial growth.

Typically, the hydrophilic group of a biosurfactant is a sugar (e.g., a mono-, di-, or polysaccharide) or a peptide, while the hydrophobic group is typically a fatty acid. Thus, there are countless potential variations of biosurfactant molecules based on, for example, type of sugar, number of sugars, size of peptides, which amino acids are present in the peptides, fatty acid length, saturation of fatty acids, additional acetylation, additional functional groups, esterification, polarity and charge of the molecule.

These variations lead to a group of molecules comprising a wide variety of classes, including, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, and high molecular weight polymers such as lipoproteins, lipopolysaccharide-protein complexes, and polysaccharide-protein-fatty acid complexes. Each type of biosurfactant within each class can further comprise subtypes having further modified structures.

Like chemical surfactants, each biosurfactant molecule has its own HLB value depending on its structure; however, unlike production of chemical surfactants, which results in a single molecule with a single HLB value or range, one cycle of biosurfactant production typically results in a mixture of biosurfactant molecules (e.g., subtypes and isomers thereof).

The phrases “biosurfactant” and “biosurfactant molecule” include all forms, analogs, orthologs, isomers, and natural and/or anthropogenic modifications of any biosurfactant class (e.g., glycolipid) and/or subtype thereof (e.g., sophorolipid).

As used herein, the term “sophorolipid,” “sophorolipid molecule,” “SLP” or “SLP molecule” includes all forms, and isomers thereof, of SLP molecules, including, for example, acidic (linear) SLP (ASL) and lactonic SLP (LSL). Further included are mono-acetylated SLP, di-acetylated SLP, esterified SLP, SLP with varying hydrophobic chain lengths, cationic and/or anionic SLP with fatty acid-amino acid complexes attached, esterified SLP, SLP-metal complexes, SLP-salt derivatives (e.g., a sodium salt of a linear SLP), and other, including those that are and/or are not described within in this disclosure.

In preferred embodiments, the SLP molecules according to the subject invention are represented by General Formula (1) and/or General Formula (2), and include 30 or more compounds having different fatty acid chain lengths (R³), and, in some instances, having an acetylation or protonation at R¹ and/or R².

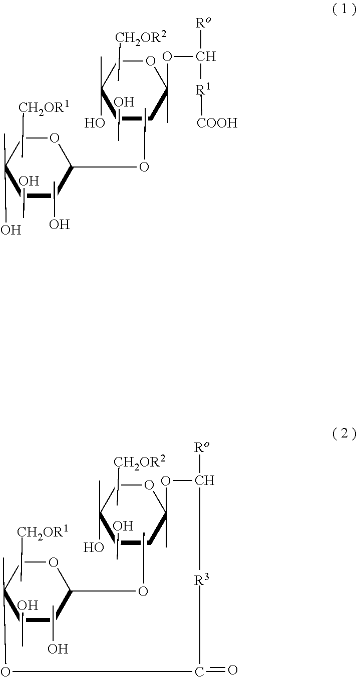

In General Formula (1) or (2), R⁰ can be either a hydrogen atom or a methyl group. R¹ and R² are each independently a hydrogen atom or an acetyl group. R³ is a saturated aliphatic hydrocarbon chain, or an unsaturated aliphatic hydrocarbon chain having at least one double bond, and may have one or more Substituents.

Examples of the Substituents include halogen atoms, hydroxyl, lower (C1-6) alkyl groups, halo lower (C1-6) alkyl groups, hydroxy lower (C1-6) alkyl groups, halo lower (C1-6) alkoxy groups, and others. R³ typically has 11 to 20 carbon atoms. In certain embodiments of the subject invention, R³ has 18 carbon atoms.

SLP are typically produced by yeasts, such as Starmerella spp. yeasts and/or Candida spp. yeasts, e.g., Starmerella (Candida) bombicola, Candida apicola, Candida batistae, Candida floricola, Candida riodocensis, Candida stellate and/or Candida kuoi. SLP have environmental compatibility, high biodegradability, low toxicity, high selectivity and specific activity in a broad range of temperature, pH and salinity conditions. Additionally, in some embodiments, SLP can be advantageous due to their small micelle size, which can help facilitate the movement of the micelle, and compounds enclosed therein, through nanoscale pores and spaces. In certain embodiments, the micelle size of a SLP is less than 100 nm, less than 50 nm, less than 20 nm, less than 15 nm, less than 10 nm, or less than 5 nm.

As used herein, “flocculation” refers to the process by which colloidal (i.e., suspended) particles form larger-sized clusters of particles by, for example, agglomerating in the form of a floc or flake.

As used herein, “beneficiation” refers to the process by which gangue materials are removed from the product of interest (e.g., element, compound, mineral).

As used herein, “ore” refers to a naturally occurring solid material from which a valuable substance, mineral and/or metal can be profitably extracted. Ores are often mined from ore deposits, which comprise ore minerals containing the valuable substance. “Gangue” minerals are minerals that occur in the deposit but do not contain the valuable substance. Examples of ore deposits include hydrothermal deposits, magmatic deposits, laterite deposits, volcanogenic deposits, metamorphically reworked deposits, carbonatite-alkaline igneous related deposits, placer ore deposits, residual ore deposits, sedimentary deposits, sedimentary hydrothermal deposits and astrobleme-related deposits. Ores, as defined herein, however, can also include ore concentrates or tailings.

As used herein, “leaching” refers to the process by which metal is extracted from ore by aqueous solutions including by, for example, ammonia leaching, alkali leaching, acid leaching, cyanidation (i.e., cyanide leaching), or thiosulfate leaching. As used herein “cyanidation” refers to the process of converting gold in ore to a water-soluble coordination complex using aqueous cyanide, including, for example, sodium cyanide, potassium cyanide, or calcium cyanide.

As used herein, “colloid” or “colloidal particle” refers to a mixture in which one insoluble substance is dispersed or suspended throughout another substance. The insoluble substance is generally dispersed in a liquid.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Flocculating Compositions

In certain embodiments, the subject invention provides compositions comprising components that are derived from microorganisms. In certain embodiments, the composition comprises a microbial biosurfactant. In certain embodiments, the composition comprises one or more biosurfactants, and, optionally, other compounds, such as, for example, water; chemical surfactants; polymers; pH adjusters; water softeners; ion exchangers; corrosion inhibitors; algaecides; biocides; clarifying agents; coagulants; inorganic salts, including, aluminum, iron, magnesium, and calcium salts; or any combination thereof.

In certain embodiments, the chemical surfactant of the flocculating composition is a detergent, wetting agent, emulsifier, foaming agent, and/or dispersant.

In certain embodiments, the polymers can include natural or synthetic polymers, water soluble polymers, cationic polymers, anionic polymers, or non-ionic polymers. The polymers can be, for example, anionic polyacrylamide, modified polyacrylamide, nonionic polyacrylamide, starch, guar gum, Moringa oleifera seed extract, Strychnos potatorum seed extract, gelatin (e.g., isinglass), alginate (e.g., sodium alginate), or chitosan.

In certain embodiments, the pH adjuster can be any compound that can yield a pH in a liquid of about 4 to about 10, about 5 to about 9, about 6 to about 8, about 6 to about 7.5, about 6.5 to about 7.5, or about 7 upon the addition of said compound. In certain embodiments, the compound is carbon dioxide, sulfuric acid, or sodium hydroxide.

In certain embodiments, the water softener is ammonia, borax, calcium hydroxide (slaked lime), trisodium phosphate, or sodium carbonate (soda ash).

In certain embodiments, the corrosion inhibitor is, for example, a chromate, nitrate, molybdate, or tungstate.

In certain embodiments, the biocide is an herbicide, insecticide, algaecide, molluscicide, miticides, piscicide, rodenticide, slimicide, germicide, antibiotic, antibacterial, antiviral, antifungal, antiprotozoal, or antiparasitic compound.

In certain embodiments, the aluminum salts can include, for example, aluminum sulfate, aluminum chloride, sodium aluminate, alum (i.e., hydrated double sulfate salt of aluminum), aluminum chlorohydrate, polyaluminum chloride, polyaluminum sulfate chloride, polyaluminum silicate chloride, and polyaluminum chloride with organic polymers; the iron salts can include ferric sulfate, ferrous sulfate, ferric chloride, ferric chloride sulfate, polyferric sulfate and ferric salts with polymers; and the magnesium salt can be magnesium carbonate or magnesium sulfate. In certain embodiments, the salts can be polymerized aluminum-iron blends. In certain embodiments, the coagulants can be lime (e.g., calcium carbonate, calcium oxide) or hydrated lime (i.e., calcium hydroxide).

In certain embodiments, the flocculating composition comprises a microbe-based product comprising a biosurfactant utilized in crude form. The crude form can comprise, in addition to the biosurfactant, fermentation broth in which a biosurfactant-producing microorganism was cultivated, residual microbial cell matter or live or inactive microbial cells, residual nutrients, and/or other microbial growth by-products. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

In some embodiments, the biosurfactant is utilized after being extracted from a fermentation broth and, optionally, purified.

The biosurfactant according to the subject invention can be a glycolipid (e.g., sophorolipids, rhamnolipids, cellobiose lipids, mannosylerythritol lipids and trehalose lipids), lipopeptide (e.g., surfactin, iturin, fengycin, arthrofactin and lichenysin), flavolipid, phospholipid (e.g., cardiolipins), fatty acid ester compound, fatty acid ether compound, and/or high molecular weight polymers such as lipoproteins, lipopolysaccharide-protein complexes, and polysaccharide-protein-fatty acid complexes.

In certain specific embodiments, the biosurfactant is a sophorolipid (SLP), including linear SLP, lactonic SLP, acetylated SLP, de-acetylated SLP, salt-form SLP derivatives, esterified SLP derivatives, amino acid-SLP conjugates, and other SLP derivatives or isomers that exist in nature and/or are produced synthetically. In preferred embodiments, the SLP is a linear SLP or a derivatized linear SLP. In certain embodiments, the subject compositions can comprise lactonic and linear SLP, with at least about 50%, about 55%, about 60%, about 65%, about 70%, about 75%, about 80%, about 85%, about 90%, or about 95% of the SLP comprising linear forms, and the remainder comprising lactonic forms.

In some embodiments, the biosurfactant can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total flocculating composition.

In another embodiment, a purified biosurfactant may be added in combination with an acceptable carrier, in that the biosurfactant may be presented at concentrations of 0.001 to 50% (v/v), preferably, 0.01 to 20% (v/v), more preferably, 0.02 to 5% (v/v).

In some embodiments, the biosurfactant can be included in the composition at, for example, 0.01 to 100,000 ppm, 0.05 to 10,000 ppm, 0.1 to 1,000 ppm, 0.5 to 750 ppm, 1.0 to 500 ppm, 2.0 to 250 ppm, or 3.0 to 100 ppm, with respect to the amount of liquid being treated.

In certain embodiments, the chemical surfactant of the flocculating composition is a detergent, wetting agent, emulsifier, foaming agent, and/or dispersant. In some embodiments, the chemical surfactant can be included in the composition at 0.01 to 99.9%, 0.1 to 90%, 0.5 to 80%, 0.75 to 70%, 1.0 to 50%, 1.5 to 25%, or 2.0 to 15% by weight, with respect to the total flocculation composition.

The flocculating composition can further comprise other additives such as, for example, carriers, other microbe-based compositions, additional biosurfactants, enzymes, catalysts, solvents, salts, buffers, chelating agents, acids, emulsifying agents, lubricants, solubility controlling agents, preservatives, stabilizers, ultra-violet light resistant agents, viscosity modifiers, preservatives, tracking agents, and other microbes and other ingredients specific for an intended use.

In certain embodiments, chelating agents can be, but are not limited to, ethylenediaminetetraacetic acid (EDTA), nitrilotriacetic acid (NTA), a phosphonate, succimer (DMSA), diethylenetriaminepentaacetate (DTPA), N-acetylcysteine, n-hydroxyethylethylenediaminetriacetic acid (HEDTA), organic acids with more than one coordination group (e.g., rubeanic acid), STPP (sodiumtripolyphosphate, Na5P3O10), trisodium phosphate (TSP), water, carbohydrates, organic acids with more than one coordination group (e.g., citric acid), lipids, steroids, amino acids or related compounds (e.g., glutathione), peptides, phosphates, nucleotides, tetrapyrrols, ferrioxamines, ionophores, orphenolics, sodium citrate, sodium gluconate, ethylenediamine disuccinic acid (EDDS), iminodisuccinic acid (IDS), L-glutamic acid diacetic Acid (GLDA), GLDA-Na₄, methyl glycindiacetic acid (MGDA), polyaspartic acid (PASA), hemoglobin, chlorophyll, lipophilic β-diketone, and (14,16)-hentriacontanedione, ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), trimethyl glycine (TMG), Tiron, or any combination thereof

### Methods of Flocculation

In certain embodiments, the subject invention provides a method for flocculating liquids from various sources, including, for example, =mining sites, quarrying sites, wastewater sites, agricultural sites, and industrial sites.

In certain embodiments, the subject invention provides a method for flocculating tailings from mines. The method comprises adding the subject compositions to the tailings and removing the agglomerated tailing particles. By flocculating the tailings, the suspended particles can reach a higher concentration when agglomerated. In preferred embodiments, the tailings are low-grade tailings, in which the tailings comprise less than about 50%, about 40%, about 35%, about 30%, or about 25% of the product of interest (e.g., metal, mineral, compound or element being mined), with the remainder comprising gangue.

In certain embodiments, the mining site can be a coal mine, iron ore mine, copper mine, copper-nickel mine, tin mine, nickel mine, gold mine, silver mine, molybdenum mine, aluminum mine (e.g., bauxite mine, kyanite mine), lead-zinc mine, tungsten mine, or zinc mine. The mine can be an underground mine, surface mine, placer mine or in situ mine. In certain embodiments, a variety of toxic compounds can be derived from mining activities. In certain embodiments, methods of removing said toxic compounds are provided according to the subject methods by contacting the flocculating compounds to various water streams, piping, pumps, water storage areas, or other aquatic environments. The toxic compounds can include, for example, cyanide, sulfur-bearing minerals, soluble iron, and heavy metals, such as, for example, molybdenum, tungsten, chromium, manganese, nickel, arsenic, and vanadium.

In certain embodiments, the microbe-containing and/or biosurfactant-containing composition can improve agglomeration between the particles or particles and a surface, such as, for example, agglomerating coal mining toxic byproducts to each other.

In certain embodiments, the microbe-containing and/or biosurfactant-containing composition can form a layer of agglomerated particulate around and/or between particles suspended in a liquid. In certain embodiments, the microbe-containing and/or biosurfactant-containing composition can neutralize charged particles.

In certain embodiments, the composition can reduce the effects of wind, rain, or physical movement of the rock, preferably in methods of reducing toxic compounds in water drainage. Examples of physical movement include tilling, digging, tunneling, excavating, transporting, dumping, crushing, blasting, separating, pelleting, cutting, casting, quarrying, dredging, fracturing, or any combination thereof.

The compositions can be applied to liquids that reside at a range of temperatures and aquatic environments, such as, for example, a stream, river, waterway, ocean, sea, lake, pond, runoff area, containment ponds, or wastewater treatment/holding tank.

The flocculating composition can be applied to a liquid and, optionally, mixed by adding, pouring, or combining.

In certain embodiments, the time period in which the flocculating composition can be contacted to a liquid is for about 1 second to about 1 year, about 1 minute to about 1 year, about 1 minute to about 6 months, about 1 minute to about 1 month, about 1 minute to about 1 week, about 1 minute to about 48 hours, about 30 minutes to 40 hours, or preferably about 12 hours to 24 hours. In certain embodiments, the methods comprise applying a liquid form of the flocculating composition to the liquid for the period of time in which liquid containing suspended particles is being produced or until the amount of suspended particles has been reduced to an amount that is determined to be satisfactory or safe, which can be readily determined by one skilled in the art. The amount of suspended particles may be considered acceptable and/or safe depending on the context. For example, the amount of suspended particles may be acceptable in higher amounts at mining sites that require personal protective equipment than in the treated water from municipal wastewater treatment facilities, in which the treated water is injected into aquifers or other waterways.

In certain embodiments, the amount of the flocculating composition applied is about 0.00001 to 15%, about 0.00001 to 10%, about 0.0001 to 5%, about 0.001 to 3%, about 0.01%, or about 1 vol % based on an amount of liquid that is treated.

In certain embodiments, the methods of the subject invention result in at least a 25% increase in flocculation of suspended particles, preferably at least a 50% increase, after one treatment. In certain embodiments, the liquid can be treated multiple times to further increase among of flocculated particles.

In certain embodiments, the flocculating composition according to the subject invention is effective due to amphiphiles-mediated adhesion of the suspended particles. In some embodiments, the sophorolipid or other biosurfactant serves as a vehicle for facilitating agglomeration of particulate matter and/or adhesion of particulate matter to a surface and/or object. For example, in some embodiments, a sophorolipid will form a micelle containing a particle, wherein the micelle is less than 1 mm, 100 μm, 50 μm, 20 μm, 10 μm, 1 μm, 100 nm, less than 50 nm, less than 25 nm, less than 15 nm or less than 10 nm in size. The small size and amphiphilic properties of the micelle allow for enhanced adhesion of the particle so that greater agglomeration of particles can occur.

In certain embodiments, the flocculating compositions can be used in methods of processing ores, ore slurries, or other products obtained via mining. In certain embodiments, the flocculating compositions can be used for dewatering before grinding, concentrate dewatering, tailings dewatering, tailings filling, middling dewatering, or any combination thereof.

In certain embodiments, the flocculating compositions can be used in beneficiation processes, particularly in low-grade ores containing low concentrations of the element or other product of interest, such as, for example, gold or silver. In order to extract the element or compound of interest, it can be necessary to crush and grind the ore and preconcentrate or separate the element or product of interest from the ore by flotation or gravity separation. In certain embodiments, the settling rate of concentrate can be accelerated, and the dewatering efficiency can be improved by adding the flocculating compositions during the beneficiation process.

In certain embodiments, the flocculating compositions can be used in methods of leaching, such as, for example, gold cyanidation. The process of extraction by leaching includes leaching (e.g., cyanide leaching), washing and filtering of leaching pulp, extraction of the metal from the leaching solution or pulp, and smelting of finished products. In certain embodiments, the flocculating compositions can be used in methods of washing and filtering leaching pulp, in which the flocculating compositions increase the rate of agglomeration of impurities. In certain embodiments, the flocculating compositions can agglomerate heavy metals and metalloids including, for example, As, Cd, Co, Cu, Hg, Mn, Ni, U, and Zn, in the mined tailings, which can present a significant potential ecological and human health risk associated with metal and metalloid exposure from contaminated soils around mined tailings storage sites.

In certain embodiments, the flocculating composition can be used in methods of treating industrial sewage and sewage water containing water-soluble organic substances. The purification process uses activated sludge treatment for the removal of soluble organic substances. In certain embodiments, activated sludge treatment uses the growth of microorganisms for processing, so a dewatering treatment of sludge is often used to reduce sludge volumes. In certain embodiments, sludge dewatering can use flocculants. In certain embodiments, the subject flocculating compositions can be used instead of synthetic polymer flocculants or in conjunction with polymer flocculants to agglomerate and dewater sludges. Cationic flocculants can neutralize negative charges on the surface of colloidal particles in sludge and can bridge between particles to form large and strong flocs for easily dewatering. Sludge containing flocs can be dehydrated by sludge-dewatering equipment, separated to solids called sludge cake, and disposed of by landfill, incineration, or compost. In certain embodiments, the flocculation methods can reduce the amount of water in the sludge by about at least about 50%, about 60%, about 70%, or about 80%.

In certain embodiments, the flocculating composition can be used in various industrial methods, including in the manufacturing or processing of food, beverages, oil sands, and paper. During the manufacturing of products, flocculating compositions can be used primarily in treating wastewater (i.e., to agglomerate a sludge), in which suspended particles, such as, for example, sludge, particles containing phosphates, and residual oil sands from oil sand tailings, are agglomerated and then removed from the liquid before the water is discharged or stored. In certain embodiments, flocculating compositions can also be used during the production process, such as, for example, during the fermentation step of beer brewing, in which yeast cells and lysed yeast cells can be agglomerated and then removed from the liquid.

Advantageously, in certain embodiments, the flocculating composition according to the subject invention provides enhanced or increased efficiency of agglomerating particles with limited negative environmental impacts. Additionally, the methods of the subject invention do not require complicated equipment or high energy consumption, and the production of the flocculating composition can be performed on site, including, for example, at a mine or at an industrial site. In certain embodiments, the subject flocculating composition can result in a decreased use of chemical surfactants, synthetic flocculants, or other potentially harmful chemicals used for flocculation.

### Production of Microbe-Based Products

In certain embodiments, the subject invention provides methods for cultivation of microorganisms and production of microbial metabolites and/or other by-products of microbial growth. The subject invention further utilizes cultivation processes that are suitable for cultivation of microorganisms and production of microbial metabolites on a desired scale. These cultivation processes include, but are not limited to, submerged cultivation/fermentation, solid state fermentation (SSF), and modifications, hybrids and/or combinations thereof.

The microorganisms can be, for example, bacteria, yeast and/or fungi. These microorganisms may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In certain embodiments, the microbes are capable of producing amphiphilic molecules, enzymes, proteins and/or biopolymers. Microbial biosurfactants, in particular, are produced by a variety of microorganisms such as bacteria, fungi, and yeasts, including, for example, Agrobacterium spp. (e.g., A. radiobacter); Arthrobacter spp.; Aspergillus spp.; Aureobasidium spp. (e.g., A. pullulans); Azotobacter (e.g., A. vinelandii, A. chroococcum); Azospirillum spp. (e.g., A. brasiliensis); Bacillus spp. (e.g., B. subtilis, B. amyloliquefaciens, B. pumillus, B. cereus, B. licheniformis, B. firmus, B. laterosporus, B. megaterium); Blakeslea; Candida spp. (e.g., C. albicans, C. rugosa, C. tropicalis, C. lipolytica, C. torulopsis); Clostridium (e.g., C. butyricum, C. tyrobutyricum, C. acetobutyricum, and C. beijerinckii); Campylobacter spp.; Cornybacterium spp.; Cryptococcus spp.; Debaryomyces spp. (e.g., D. hansenii); Entomophthora spp.; Flavobacterium spp.; Gordonia spp.; Hansenula spp.; Hanseniaspora spp. (e.g., H. uvarum); Issatchenkia spp; Kluyveromyces spp.; Meyerozyma spp. (e.g., M guilliermondii); Mortierella spp.; Mycorrhiza spp.; Mycobacterium spp.; Nocardia spp.; Pichia spp. (e.g., P. anomala, P. guilliermondii, P. occidentalis, P. kudriavzevii); Phycomyces spp.; Phythium spp.; Pseudomonas spp. (e.g., P. aeruginosa, P. chlororaphis, P. putida, P. florescens, P. fragi, P. syringae); Pseudozyma spp. (e.g., P. aphidis); Ralslonia spp. (e.g., R. eulropha); Rhodococcus spp. (e.g., R. erythropolis); Rhodospirillum spp. (e.g., R. rubrum); Rhizobium spp.; Rhizopus spp.; Saccharomyces spp. (e.g., S. cerevisiae, S. boulardii sequela, S. torula); Sphingomonas spp. (e.g., S. paucimobilis); Starmerella spp. (e.g., S. bombicola); Thraustochytrium spp.; Torulopsis spp.; Ustilago spp. (e.g., U. maydis); Wickerhamomyces spp. (e.g., W. anomalus); Williopsis spp.; and/or Zygosaccharomyces spp. (e.g., Z. bailii).

In preferred embodiments, microorganism is a Starmerella spp. yeast and/or Candida spp. yeast, e.g., Starmerella (Candida) bombicola, Candida apicola, Candida batistae, Candida floricola, Candida riodocensis, Candida stellate and/or Candida kuoi. In a specific embodiment, the microorganism is Starmerella bombicola, e.g., strain ATCC 22214.

As used herein “fermentation” refers to cultivation or growth of cells under controlled conditions. The growth could be aerobic or anaerobic. In preferred embodiments, the microorganisms are grown using SSF and/or modified versions thereof.

In one embodiment, the subject invention provides materials and methods for the production of biomass (e.g., viable cellular material), extracellular metabolites (e.g., small molecules and excreted proteins), residual nutrients and/or intracellular components (e.g., enzymes and other proteins).

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, humidity, microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique. Dilution plating is a simple technique used to estimate the number of organisms in a sample. The technique can also provide an index by which different environments or treatments can be compared.

In one embodiment, the method includes supplementing the cultivation with a nitrogen source. The nitrogen source can be, for example, potassium nitrate, ammonium nitrate ammonium sulfate, ammonium phosphate, ammonia, urea, and/or ammonium chloride. These nitrogen sources may be used independently or in a combination of two or more.

The method can provide oxygenation to the growing culture. One embodiment utilizes slow motion of air to remove low-oxygen containing air and introduce oxygenated air. In the case of submerged fermentation, the oxygenated air may be ambient air supplemented daily through mechanisms including impellers for mechanical agitation of liquid, and air spargers for supplying bubbles of gas to liquid for dissolution of oxygen into the liquid.

The method can further comprise supplementing the cultivation with a carbon source. The carbon source is typically a carbohydrate, such as glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, canola oil, rice bran oil, olive oil, corn oil, sesame oil, and/or linseed oil; etc. These carbon sources may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, and microelements can be included, for example, in the form of flours or meals, such as corn flour, or in the form of extracts, such as yeast extract, potato extract, beef extract, soybean extract, banana peel extract, and the like, or in purified forms. Amino acids such as, for example, those useful for biosynthesis of proteins, can also be included.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, sodium chloride, calcium carbonate, and/or sodium carbonate. These inorganic salts may be used independently or in a combination of two or more.

In some embodiments, the method for cultivation may further comprise adding additional acids and/or antimicrobials in the medium before, and/or during the cultivation process. Antimicrobial agents or antibiotics are used for protecting the culture against contamination.

Additionally, antifoaming agents may also be added to prevent the formation and/or accumulation of foam during submerged cultivation.

The pH of the mixture should be suitable for the microorganism of interest. Buffers, and pH regulators, such as carbonates and phosphates, may be used to stabilize pH near a preferred value. When metal ions are present in high concentrations, use of a chelating agent in the medium may be necessary.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method for cultivation of microorganisms is carried out at about 5° to about 100° C., preferably, 15 to 60° C., more preferably, 25 to 50° C. In a further embodiment, the cultivation may be carried out continuously at a constant temperature. In another embodiment, the cultivation may be subject to changing temperatures.

In one embodiment, the equipment used in the method and cultivation process is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Air can be sterilized by methods know in the art. For example, the ambient air can pass through at least one filter before being introduced into the vessel. In other embodiments, the medium may be pasteurized or, optionally, no heat at all added, where the use of low water activity and low pH may be exploited to control undesirable bacterial growth.

In one embodiment, the subject invention further provides a method for producing microbial metabolites such as, for example, biosurfactants, enzymes, proteins, ethanol, lactic acid, beta-glucan, peptides, metabolic intermediates, polyunsaturated fatty acid, and lipids, by cultivating a microbe strain of the subject invention under conditions appropriate for growth and metabolite production; and, optionally, purifying the metabolite. The metabolite content produced by the method can be, for example, at least 20%, 30%, 40%, 50%, 60%, 70%, 80%, or 90%.

The microbial growth by-product produced by microorganisms of interest may be retained in the microorganisms or secreted into the growth medium. The medium may contain compounds that stabilize the activity of microbial growth by-product.

The biomass content of the fermentation medium may be, for example, from 5 g/l to 180 g/l or more, or from 10 g/l to 150 g/l.

The cell concentration may be, for example, at least 1×10⁶ to 1×10¹², 1×10⁷ to 1×10¹¹, 1×10⁸ to 1×10¹⁰, or 1×10⁹ CFU/ml.

The method and equipment for cultivation of microorganisms and production of the microbial by-products can be performed in a batch, a quasi-continuous process, or a continuous process.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells, spores, conidia, hyphae and/or mycelia remains in the vessel as an inoculant for a new cultivation batch. The composition that is removed can be a cell-free medium or contain cells, spores, or other reproductive propagules, and/or a combination of thereof. In this manner, a quasi-continuous system is created.

Advantageously, the method does not require complicated equipment or high energy consumption. The microorganisms of interest can be cultivated at small or large scale on site and utilized, even being still-mixed with their media.

In certain embodiments, the subject invention provides a “microbe-based composition,” meaning a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, expressed proteins, and/or other cellular components. The microbes may be intact or lysed. The microbes may be present in or removed from the composition. The microbes can be present, with broth in which they were grown, in the microbe-based composition. The cells may be present at, for example, a concentration of at least 1×10³, 1×10⁴, 1×10⁵, 1×10⁶, 1×10⁷, 1×10⁸, 1×10⁹, 1×10¹⁰, 1×10¹¹, 1×10¹², 1×10¹³ or more CFU per milliliter of the composition.

The subject invention further provides “microbe-based products,” which are products that are to be applied in practice to achieve a desired result. The microbe-based product can be simply a microbe-based composition harvested from the microbe cultivation process. Alternatively, the microbe-based product may comprise further ingredients that have been added. These additional ingredients can include, for example, stabilizers, acids, buffers, carriers, such as water, salt solutions, or any other appropriate carrier, added nutrients to support further microbial growth, non-nutrient growth enhancers, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

One microbe-based product of the subject invention is simply the fermentation medium containing the microorganisms and/or the microbial metabolites produced by the microorganisms and/or any residual nutrients. The product of fermentation may be used directly without extraction or purification. If desired, extraction and purification can be easily achieved using standard extraction and/or purification methods or techniques described in the literature.

The microorganisms in the microbe-based products may be in an active or inactive form, or in the form of vegetative cells, reproductive spores, conidia, mycelia, hyphae, or any other form of microbial propagule. The microbe-based products may also contain a combination of any of these forms of a microorganism.

In one embodiment, different strains of microbe are grown separately and then mixed together to produce the microbe-based product. The microbes can, optionally, be blended with the medium in which they are grown and dried prior to mixing.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

Upon harvesting the microbe-based composition from the growth vessels, further components can be added as the harvested product is placed into containers or otherwise transported for use. The additives can be, for example, buffers, carriers, other microbe-based compositions produced at the same or different facility, viscosity modifiers, preservatives, nutrients for microbe growth, surfactants, emulsifying agents, lubricants, solubility controlling agents, tracking agents, solvents, biocides, antibiotics, pH adjusting agents, chelators, stabilizers, ultra-violet light resistant agents, other microbes and other suitable additives that are customarily used for such preparations.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

1. A method of flocculation, the method comprising contacting a flocculating composition comprising a glycolipid biosurfactant with a liquid containing suspended particles to yield an agglomerated particle.

2. The method of claim 1, further comprising removing said agglomerated particle from the liquid.

3. The method of claim 1, wherein the agglomerated particle comprises a toxic substance or gangue.

4. The method of claim 3, wherein the toxic substance is cyanide, a sulfur-bearing mineral, soluble iron, molybdenum, tungsten, chromium, manganese, nickel, arsenic, or vanadium.

5. The method of claim 1, wherein the liquid is wastewater or a slurry of ore.

6. The method of claim 5, wherein the wastewater is municipal wastewater, stormwater, mining wastewater, quarrying wastewater, or industrial wastewater.

7. The method of claim 5, wherein the wastewater or slurry of ore is from a coal mine, iron ore mine, copper mine, copper-nickel mine, tin mine, nickel mine, gold mine, silver mine, molybdenum mine, aluminum mine, lead-zinc mine, tungsten mine, or zinc mine.

8. The method of claim 7, wherein the aluminum mine is a kyanite mine or a bauxite mine.

9. The method of claim 1, wherein the flocculating composition further comprises a chemical surfactant, polymer, pH adjuster, water softener, ion exchanger, corrosion inhibitor, biocide, clarifying agent, coagulant, inorganic salt, or any combination thereof.

10. (canceled)

11. The method of claim 1, wherein the glycolipid biosurfactant is a sophorolipid and/or a yeast culture comprising a sophorolipid.

12. The method of claim 11, wherein the yeast culture is a Starmerella sp. and/or a Candida sp. yeast.

13. The method of claim 11, wherein the yeast is in a vegetative state.

14. The method of claim 11, wherein the yeast is in a spore form.

15. The method of claim 1, wherein the flocculation comprises agglomeration of colloidal particles by one or a combination of the following:
a) adhering particles to a surface and/or object;
b) agglomerating particles together;
c) settling particles; or
d) neutralizing particles.

16. A flocculating composition comprising a sophorolipid and/or a yeast culture comprising a sophorolipid and one or more traditional flocculating components.

17. The composition of claim 16, wherein the yeast culture is a Starmerella sp. and/or a Candida sp. yeast.

18. The composition of claim 16, wherein the yeast is in a vegetative state.

19. The composition of claim 16, wherein the yeast is in a spore form.

20. The composition of claim 16, wherein the traditional flocculating components are selected from one or a combination of the following:
a) chemical surfactant;
b) polymer;
c) pH adjuster;
d) water softener;
e) ion exchanger;
f) corrosion inhibitor;
g) biocide;
h) clarifying agent;
i) coagulant; and
j) inorganic salt.

21. The composition of claim 20, wherein the inorganic salt is aluminum sulfate, aluminum chloride, sodium aluminate, alum, aluminum chlorohydrate, polyaluminum chloride, polyaluminum sulfate chloride, polyaluminum silicate chloride, polyaluminum chloride with organic polymers, ferric sulfate, ferrous sulfate, ferric chloride, ferric chloride sulfate, polyferric sulfate, ferric salts with polymers, magnesium carbonate, magnesium sulfate, polymerized aluminum-iron blends, lime, or any combination thereof.
